(19) United States
(12) Patent Application Publication
(10) Pub. No.: US 2004/0074293 A1
(43) Pub. Date: Apr. 22, 2004

(54) Method of determining the adhesion properties of materials

(76) Inventors: Astrid Pundt, Goettingen (DE); Reiner Kirchheim, Goettingen (DE)

Correspondence Address: NATH & ASSOCIATES PLLC, Sixth Floor, 1030 15th Street, N.W., Washington, DC, 20005, US

(21) Appl. No.: 10/615,453

(22) Filed: Jul. 9, 2003

(30) Foreign Application Priority Data
Jul. 9, 2002 (DE) 102 31 383.0

(51) Int. Cl. G01N 19/04; G01N 33/00; G01N 33/20; G01N 33/44

(52) U.S. Cl. 73/150 A; 73/866

(57) ABSTRACT

In a method of determining the adhesion properties of at least two materials to one another at least one material is charged with an absorbate and a critical parameter is determined at which the materials undergo at least partial, in particular substantially complete, detachment from one another. Films/coatings on substrates are investigated with preference. By evaluating the results it is possible, for example, to give a quantitative determination of adhesion energies. This allows predictions about the adhesion of different materials to/on one another.

The invention relates to a method of determining the adhesion properties of at least two materials to one another, in particular of determining the adhesion properties of at least one coating or film on a substrate.

The question of whether two materials adhere firmly to one another sufficiently or at all is of great importance in many areas of industry. Just two examples of this are, first, the adhesion of polymer films, especially coating materials, to metals or plastics, in the car industry, for example, and, secondly, the adhesion of metal films to polymers, in the foodstuffs industry, for example (milk cartons, crisp bags and the like). Insufficient adhesion between the materials chosen results in detachment of the materials from one another. Stresses resulting in detachment often result from different thermal expansion coefficients of said materials, from oxidation effects or from other mechanisms. Such detachment is unacceptable for an industrial application. In that case it is necessary to find a more suitable combination of materials, a task which may involve much effort.

For testing the adhesion properties of different materials to one another a variety of methods are known from the prior art. One example is the “pull-off” test, in which an adhesively applied die is pulled with a defined force perpendicularly to the bonded materials. Also known is the adhesive tape test, in which an adhesive strip is applied to the bonded materials and subsequently removed. Finally there are also scratch tests, in which a pointed article, such as a needle, scratches a material with a defined force. A modification of such techniques is the indentation test, in which a sharp point is pressed into the material and the edges which form, or the shape which forms when the point is withdrawn, are subjected to optical analysis. In addition it is also possible to employ other processes, such as scanning probe techniques or acoustic emission techniques, in order to ascertain and investigate instances of delamination.

The processes specified have a variety of disadvantages. Thus in the case of the adhesive tape test or pull-off test the investigation covers material properties of the adhesive or adhesive strip which have nothing to do with the actual adhesion of the materials to one another. Furthermore, these tests, and the above-described scratch techniques as well, are not non-destructive in their operation and are often difficult to reproduce. The other specified tests necessitate comparatively complicated and expensive apparatus. All of the said processes have the disadvantage, moreover, that reliable quantification of the adhesion properties is not possible.

It is additionally known that the absorption of an absorbate in layer systems and film systems can lead to the detachment of these layers and films. The absorption of the absorbate, for example a gas such as hydrogen, produces mechanical stresses in the layer, which can lead ultimately to the detachment of that layer. By way of literature, reference may be made here to the publication by U. Laudahn et al. in Journal of Alloys and Compounds, 293-295 (1999), 490-494. Those investigations, however, do not go beyond the simple observation of the induced mechanical stresses.

The object addressed by the invention is reliably and reproducibly to determine the adhesion properties of at least two materials to one another. The corresponding method is to be capable of being applied in particular for determining the adhesion properties of coatings or films on a substrate. The method ought preferably to give access to quantifiable results, so that layer systems can be tested prior to their industrial application for the respective requirements in terms of adhesion properties.

This object is achieved by the method having the features of claim 1. Preferred embodiments of the method are set out in the dependent claims 2 to 26. The wording of all the claims is hereby made part of the content of this description, by reference.

In accordance with the invention the method referred to at the outset takes a form in which at least one material, preferably the coating or film, is charged with at least one absorbate. Then at least one critical physical or chemical parameter is determined at which the two materials part from one another at least partially, in particular substantially completely. As a result of the absorption of the absorbate into one of the two materials, which takes place in a controlled manner in the context of the invention, that material swells or expands. The mechanical stresses which develop as a result of its adhesion to the other material lead to the at least partial detachment of the materials from one another. In this way it is possible to determine a critical physical or chemical parameter which serves as a measure of the adhesion properties of the two materials to one another.

In one preferred embodiment of the method of the invention the critical parameter is the amount of absorbate with which one of the two materials, in particular the coating or film, is charged. As elucidated further below, it is frequently easy to determine such amounts of absorbate.

In another preferred embodiment of the method of the invention the critical parameter is the curvature of a material, preferably of the substrate, which results from the charging of this material with the absorbate. Curvatures of this kind are also often easy to determine. This will be elucidated further below, in context with the example.

It will be appreciated that the invention is not restricted to the two specified critical parameters (amount of absorbate, substrate curvature). It is possible in principle to choose any parameters which can be determined in the event of partial or complete detachment of the two materials from one another.

In principle it is possible in accordance with the invention to determine the critical physical or chemical parameter with single implementation of the method steps. This is the case in particular when the initial stresses for a chosen combination of materials are negligible. In that case only one determination/measurement per combination of materials is necessary in order to determine the adhesion properties. In accordance with the invention it is preferred, however, if the method steps are implemented at least twice, in particular multiply. In that case at least one factor of the material, in particular the thickness of the coating or film, is varied. The fewer the number of times it is necessary to implement the method steps, the quicker the adhesion properties can be determined for a chosen combination of materials. Accordingly it is further preferred if the method steps in question are implemented only twice per combination of materials.

In the method of invention, with the aid of the critical parameter obtained, it is preferred to determine (arithmetically) the quantity referred to as adhesion energy. In this case the corresponding mathematical function can be derived in accordance with the materials used and the specific objective. In this case the adhesion energy is determined in particular by determining the slope of a plot of the critical parameter against a function, preferably the reciprocal root of the material factor. The material factor is preferably the thickness of the coating or film. The adhesion energy obtained is a direct measure of the adhesiveness of the two materials to one another or on one another. The precise procedure associated with these preferred embodiments of the invention is evident from the further description and from the figures.

The advantages of the method of the invention are manifested in particular in the case of assembled material systems in which the thickness of the first material, in particular of the coating or film, is low relative to the thickness of the second material, in particular of the substrate. This corresponds exactly to the preferred applications where adhesion properties of at least two materials to one another or on one another are to be determined. Assembled material systems for which the method of the invention can be employed with particular preference can be defined by the relationship Es²/d, where E is the elasticity modulus of the substrate, s is the thickness of the substrate, and d is the thickness of the coating or film. Corresponding preferred values for this relationship lie between 10⁸ Pa·m and 10¹⁴ Pa·m, in particular between 10¹⁰ Pa·m and 10¹³ Pa·m.

In principle it is possible to use the method of the invention to investigate assembled material systems featuring very different material factors. It is preferred, however, if the material factors, i.e. the thickness both of the first material and of the second material, in particular of the coating/film and of the substrate, respectively, are between 1 nm (nanometer) and 5 mm (millimetres). Within these ranges mention may be made, for the thickness of the substrate, of values between 1 μm (micrometer) and 5 mm and, for the thickness of the coating or film, of values between 1 nm and 1 μm. These values specified with preference also relate to the preferred applications for the determination of adhesion properties.

The absorbate with which at least one material is charged is preferably at least one liquid. In the case of a first group of further-preferred embodiments this liquid is water. In the case of another group of preferred embodiments the liquid is an organic solvent, in particular dichloromethane or tetrachloromethane.

In other particularly preferred embodiments of the invention the absorbate is at least one gas. Mention may be made here as preferred gases of carbon dioxide or gaseous elements, in particular hydrogen. Hydrogen specifically can be incorporated into materials in a simple and reproducible way and in defined amounts.

In principle the charging of the material with the absorbate in accordance with the invention may take place in any desired way. However, it is preferred, on the one hand, if the charging with the absorbate takes place directly from the liquid phase or from the gas phase. On the other hand, particular preference is given to embodiments in which the charging with a gaseous absorbate, in particular with hydrogen, takes place by means of what is known as electrochemical charging. In this procedure the gas, in particular the hydrogen, is generated directly on the material to be charged, electrochemically, by charge flux, for example from hydrogen ions in preferably acidic solutions. This is elucidated in more detail in connection with the figures.

In accordance with the invention the first material, in particular the material of the coating or film, is preferably a metal, in particular a noble metal. In further preferred embodiments of the method of the invention the first material, in particular the material of the coating or film, is a polymer material, in particular a coating material.

As the second material, in particular as the substrate, use is made preferably, in accordance with the invention, of a polymer material. These embodiments relate accordingly in particular to the combination of materials represented by a coating/film of metal on a substrate of polymer material (plastic).

Likewise preferred in accordance with the invention are embodiments in which a metal is used preferably as second material, in particular as a substrate. These embodiments comprise the preferred combinations of materials represented by a coating/film of polymer material (e.g. coating material) on a metal substrate.

The determination of the critical parameter during the partial, in particular substantially complete, detachment of the two materials from one another can take place in principle in any desired way. The critical parameter is preferably determined at the moment when locally a detachment of the two materials from one another takes place for the first time, for example by formation of a dome or hummock. In preferred embodiments of the invention the process of detachment of the two materials (particularly detachment of coating/film from substrate) is monitored with the aid of optical processes. In the simplest case this can be done visually, in particular with the aid of a light microscope or with a corresponding, different optical device (e.g. CCD camera).

Another preferred procedure comprises the determination of the surface roughness, or of the change therein, during the detachment process. This surface roughness can likewise be determined in any desired way, the use of optical methods being preferred here as well. Thus it is possible in particular to determine the surface reflectivity or the surface scattering behaviour, or the change in these variables.

The method of the invention can also be employed for combinations of materials in which the two materials of the assembly are themselves unable to absorb any absorbate or are able to absorb only a little absorbate. In these cases at least one coating or corresponding film which absorbs the absorbate is firmly connected to a further coating/film which does not absorb the absorbate, or which absorbs it only at a low concentration. Then, by charging the absorbate-accepting coating/film with the absorbate, the adhesion properties of the coating/film which absorbs the substrate only at a low concentration, or not at all, to the substrate are determined. This procedure is illustrated further in connection with the figures.

From the depiction of the last-described preferred embodiments it is evident that the invention is not restricted to the investigation of combinations of two materials. For the determination of the adhesion properties preference is given to a layer construction (including the substrate) of from two to four, in particular two or three, layers.

The method of the invention has the advantage that through the controlled charging of material in a layer construction it is possible to determine the adhesion properties of two materials to one another or on one another in a simple and reproducible way. If the concentration of absorbate is deliberately raised to a point where there is only partial detachment of the materials from one another, the determination of the adhesion properties is quasi-non-destructive. The conclusions obtained by means of the method of the invention make it possible to investigate the adhesion properties of material systems in a defined way at an early stage. Also possible are quantitative conclusions, allowing targeted prior selection of certain combinations of materials.

The features described, and further features of the invention, are evident from the following description of preferred embodiments in conjunction with the dependent claims and with the drawings. In the context of these embodiments the individual features may each be actualized individually or in combinations of two or more of them with one another.

In the drawings:

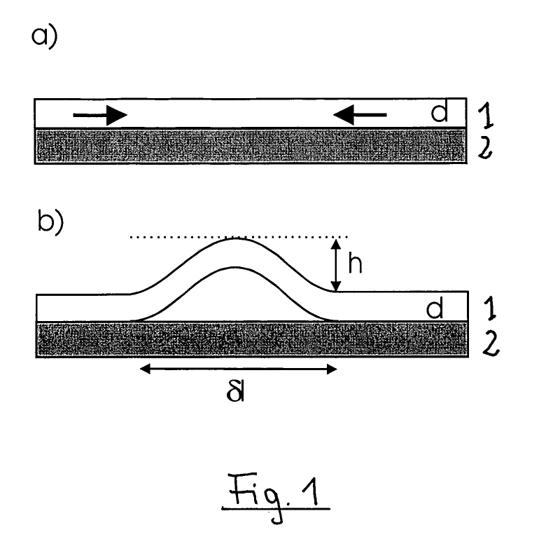

FIG. 1 shows the schematic representation of an assembly system of two materials during the absorption of the absorbate into the upper of the two materials,

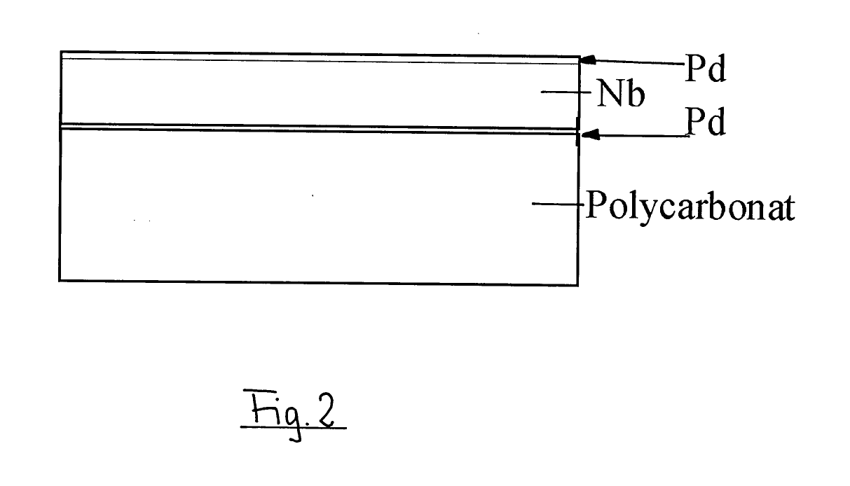

FIG. 2 shows the layer construction of the embodiments of the method of the invention that are elucidated in the example,

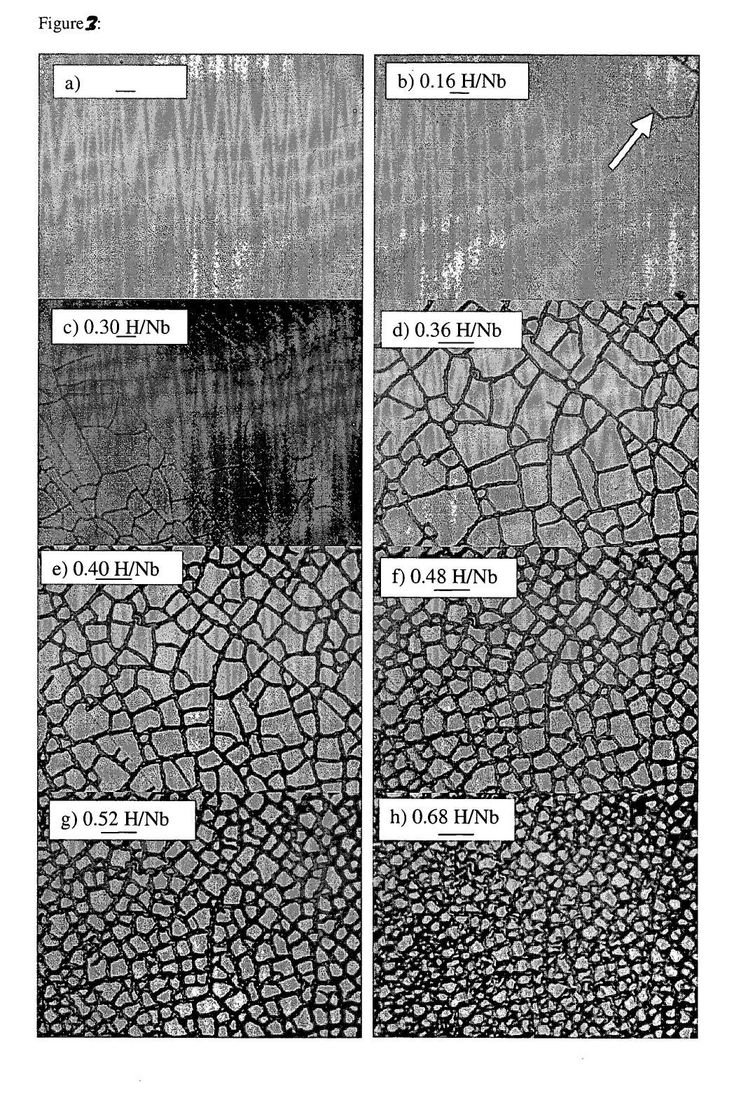

FIG. 3 shows the morphological development of the surface of the layer construction of the embodiment of the method of the invention that is elucidated in the example, and

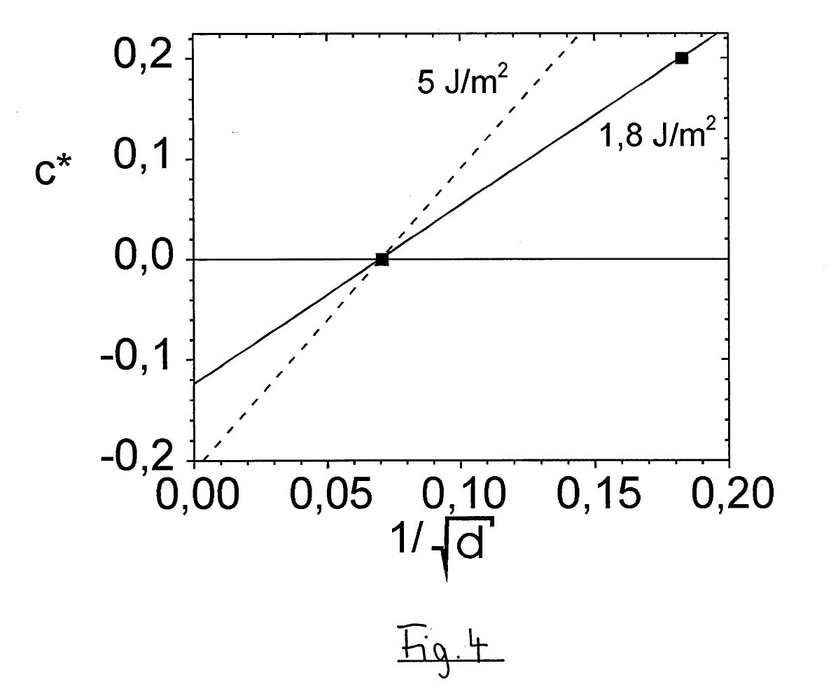

FIG. 4 shows the determination of the adhesion energy in the case of the embodiment of the method of the invention that is elucidated in the example.

In FIG. 1 the principle on which the method of the invention is based is elucidated schematically with a simple representation.

Two materials 1 and 2 adhere to one another and in so doing form an assembly system. The absorption (not depicted in detail) in FIG. 1a) of an absorbate, for example hydrogen, into the top layer (material 1) generates high stresses, depicted by the arrows, in that layer. This leads in accordance with FIG. 1b) to a detachment of the top layer (material 1) from the bottom layer/substrate (material 2) over the length δl depicted in FIG. 1b). Over this length the adhesion to material 1 and material 2 is lost, with the maximum height h of the detached portion of the material 1 (as depicted in FIG. 1b)).

### EXAMPLE

Thin palladium/niobium/palladium layer sequences are deposited by argon sputtering on polycarbonate substrates 250 μm thick. For this purpose the polycarbonate substrates are firstly briefly cleaned with isopropanol. The metal films are produced at room temperature in an ultra-high-vacuum chamber (10⁻¹⁰ mbar) under an argon gas pressure of 10⁻⁴ mbar. This produces the layer construction depicted in FIG. 2.

In the present case the thicknesses of the palladium layers located above and below the niobium layer are in each case 10 nm. The top palladium layer serves to prevent the oxidation of the niobium layer and to facilitate the absorption of hydrogen (absorbate=hydrogen) into the niobium layer. It is within the niobium layer that the internal stresses are to be generated, so that the palladium layer disposed below the niobium layer detaches itself from the polycarbonate substrate. The adhesion properties under investigation, therefore, are those of the (bottom) palladium layer to the polycarbonate substrate. In the example niobium layers of 10 nm, 30 nm, 100 nm and 200 nm in thickness are produced with low deposition rates of 1.6 nm/min.

All of the layer constructions obtained are investigated under a light microscope before being charged with hydrogen. They have substantially smooth metal surfaces with just a few scratches, which originate from the polycarbonate substrate.

Subsequently the layer construction is electrochemically charged with hydrogen through the top palladium layer (which serves, so to speak, as a “window”). For this purpose a solution of glycerol and phosphoric acid (2:1) is used. This electrochemical charging represents a simple process, since the hydrogen concentration absorbed into the layer construction can be determined easily by measurement of the electrical charge, using Faraday's law (R. Kirchheim, Prog. Mat. Sci. 32 (1988), 261-325). The literature reference cited also depicts the construction of corresponding apparatus. In order to be able to monitor the absorption of the hydrogen, the sample in question was constructed on the frame of an optical microscope. The change in surface structure in the course of charging with hydrogen was monitored optically in situ using a CCD camera.

The results of this optical monitoring are depicted in FIG. 3. This shows the pictures from the CCD camera during the hydrogen charging of a) 0 H/Nb up to h) 0.68H/Nb. The bar shown beneath the concentration figures corresponds in each case to a length of 100 μm. FIG. 3 represents the result of the method of invention for a niobium layer thickness of 100 nm.

As FIG. 3 shows, to start with (0 H) only a few scratches originating from the substrate can be seen. By charging with hydrogen and absorption of the hydrogen into the niobium layer by way of the topmost palladium layer the stresses in the layer plane of the niobium are gradually increased. The surface of the layer construction remains substantially unchanged up to a hydrogen concentration of 0.14 H/Nb. Above this concentration additional linear lines appear, as indicated by the arrow in FIG. 3b). It is here, in other words, that the initial formation of cracks occurs. By changing the illumination conditions in the light microscope and by additional experiments in an interference microscope it was found that here in actual fact there is a lifting of the surface. As the hydrogen concentrations are increased further the lines extend further over the entire surface, as depicted in FIGS. 3d) to 3h). Additionally there is an increase in the width of the lines in tandem with the hydrogen concentration. Finally a net-shaped surface structure is obtained, which grows finer and finer as the hydrogen concentration increases. By means of optical monitoring a critical hydrogen concentration is determined at which the top layers first undergo local detachment from the polycarbonate substrate. In the present case, it is the first visible doming which is used for determining this critical concentration.

Film detachment and the patterns which appear are investigated for different niobium film thicknesses, leading to comparable optical results as in FIG. 3. Here again a measurement is made of the critical hydrogen concentrations at which the top layers first underwent local detachment from the substrate. Relatively thin niobium layers absorb comparatively higher concentrations of hydrogen before film detachment commences and the corresponding net-shaped domes are formed.

The evaluation of the results obtained for different layer thicknesses is depicted in FIG. 4. This a plot of the critical absorbate concentration (designated c*) as a function of the reciprocal thickness of the material 1/√d (d=layer thickness of the niobium). The slope of this plot, namely \sqrt{(1−v)·γ/(α_{H}²E)}, where v is the Poisson number of niobium, α_{H} is the material expansion constant of niobium, E is the elasticity modulus of niobium, gives the adhesion energy γ. This is the measure of the adhesion of two materials to one another. In the case of the example under discussion here the adhesion energy of palladium (bottom layer in FIG. 2) and polycarbonate substrate was found to be 1.8 J/m². Inserted merely for comparison in FIG. 3 is the course of a straight line which would correspond to an adhesion energy of 5 J/m².

In a comparable manner the adhesion energy can be determined by measuring the curvature of the substrate with a suitable strain gauge. The stresses occurring in the niobium layer during loading with hydrogen are determined by the curvature of the substrate, said substrate e.g. being clamped firmly at one side in a suitable apparatus. The curvature e.g. can be detected with an inductive strain gauge which senses a metal piece attached to the other side of the substrate. The surface of the layer system during the loading with hydrogen and the resulting domes and detachments are also monitored with an optical microscope and a CCD camera. In this case the critical parameter is the curvature of the substrate, from which a critical stress value in the niobium layer can be calculated, when the top layers detach from the substrate. As an alternative, the critical stress also can be determined as the stress value, at which in a plot of stress as a function of hydrogen concentration in the niobium layer, the measured stress values deviate significantly, e.g. by 10%, from the extrapolated linear behaviour of such plots at low hydrogen concentrations. When such critical stress values are plotted as a function of the reciprocal thickness of the material 1/√d (d=layer thickness of the niobium), the adhesion energy (here: of palladium) can be calculated also.

The remarks above show that the method of the invention is suitable for determining, with comparative simplicity, the adhesion properties of two materials to one another or on one another. By way of the critical parameter it is possible quantitatively to obtain values, for example for the adhesion energy, so that predictions are possible about the adhesion of different materials to/on one another. This renders the method of the invention of interest for many technical applications in which such predictions are desired.

1. Method of determining the adhesion properties of at least two materials to one another, in particular of determining the adhesion properties of at least one coating or film of at least one first material to a substrate of a second material, wherein at least one material, preferably at least one coating or film, is charged with at least one absorbate and at least one critical physical or chemical parameter is determined at which the materials, preferably at least one coating or film, detach from one another, preferably from the substrate, at least partially, in particular substantially completely.

2. Method according to claim 1, wherein the critical parameter is the amount of absorbate charged.

3. Method according to claim 1, wherein the critical parameter is the substrate curvature resulting where appropriate from charging with the absorbate.

4. Method according to claim 1, wherein the method steps are implemented at least twice, in particular multiply, and at least one material factor, in particular the thickness of the coating or film, is varied.

5. Method according to claim 1, wherein the critical parameter is used to determine the quantity known as the adhesion energy.

6. Method according to claim 5, wherein the adhesion energy is determined by determining the slope of a plot of the critical parameter against a function, preferably the reciprocal root, of the material factor, in particular the thickness of the coating or film.

7. Method according to claim 1, wherein the thickness of the coating or film is low relative to the thickness of the substrate.

8. Method according to claim 1, wherein the relationship Es²/d is between 10⁸ Pa·m and 10¹⁴ Pa·m, preferably between 10¹⁰ Pa·m and 10¹³ Pa·m, where E is the elasticity modulus of the substrate, s is the thickness of the substrate and d is the thickness of the coating or film.

9. Method according to claim 1, wherein the material factor, in particular the thickness of the coating or film, and/or of the substrate is between 1 nm and 5 mm.

10. Method according to claim 9, wherein the thickness of the substrate is between 1 μm and 5 mm and the thickness of the coating or film is between 1 nm and 1 μm.

11. Method according to claim 1, wherein the absorbate is at least one liquid.

12. Method according to claim 11, wherein the absorbate is water.

13. Method according to claim 11, wherein the absorbate is an organic solvent, in particular dichloromethane or tetrachloromethane.

14. Method according to claim 1, wherein the absorbate is at least one gas.

15. Method according to claim 14, wherein the absorbate is carbon dioxide.

16. Method according to claim 14, wherein the absorbate is a gaseous element, in particular hydrogen.

17. Method according to claim 1, wherein charging with the absorbate takes place directly from the liquid phase or gas phase.

18. Method according to claim 1, wherein charging with the gas takes place by electrochemical charging.

19. Method according to claim 1, wherein the first material, in particular the material of the coating or film, is a metal, in particular a noble metal.

20. Method according to claim 1, wherein the first material, in particular the material of the coating or film, is a polymer material, in particular a coating material.

21. Method according to claim 1, wherein the second material, in particular the substrate, is a polymer material.

22. Method according to claim 1, wherein the second material, preferably the substrate, is a metal.

23. Method according to claim 1, wherein in order to determine the critical parameter the detachment process of the two materials, in particular of the coating or film from the substrate, is monitored optically, in particular using a light microscope.

24. Method according to claim 1, wherein in order to determine the critical parameter in the detachment process of the two materials, in particular of the coating or film from the substrate, the surface roughness is monitored, in particular by determination of the surface reflectivity and/or of the surface scattering behaviour.

25. Method according to claim 1, wherein at least one coating/film which absorbs the absorbate is firmly connected to at least one further coating/film which does not absorb the absorbate, or which absorbs it only at a low concentration, and by charging the coating/film which absorbs the absorbate with the absorbate the adhesion properties of the coating/film which does not absorb the absorbate or which absorbs it only at a low concentration to the substrate is determined.

26. Method according to claim 1, wherein for the determination of the adhesion properties, including the substrate, a layer construction of from two to four, preferably two or three, layers is provided.
